(19) United States
(12) Patent Application Publication
(10) Pub. No.: US 2020/0232974 A1
(43) Pub. Date: Jul. 23, 2020

(54) LYMPHOCYTES EXPRESSING CD73 IN CANCEROUS PATIENT DICTATES THERAPY

(71) Applicant: Centre Léon-Bérard, LYON (FR)

(72) Inventors: Christine MENETRIER-CAUX, BRESSOLLES (FR); Nicolas GOURDIN, COULOGNE (FR); Christophe CAUX, BRESSOLLES (FR); Selena VIGANO, LAUSANNE (CH); Pedro ROMERO, EPALINGES (CH); Marion BOSSENEC, LYON (FR)

(21) Appl. No.: 16/090,006

(22) PCT Filed: Mar. 30, 2017
(86) PCT No.: PCT/EP2017/057611

§ 371 Date: Sep. 28, 2018

(30) Foreign Application Priority Data
Mar. 30, 2016 (EP) 16305369.7

(51) Int. Cl. G01N 33/50; G01N 33/574

(52) U.S. Cl. CPC G01N 33/5091; G01N 2333/916; G01N 33/57488

(57) ABSTRACT

The invention discloses a method for selecting a human patient for CD73 antagonist therapy, preferably immunotherapy, comprising the step of determination in a patient's biological sample, that lymphocytes, preferably CD4+ T cells, express CD73, wherein, upon a determination that lymphocytes, preferably CD4+ T cells, express CD73 in patient's biological sample, the patient is declared sensitive to immunotherapy using an antagonist of CD73, preferably an anti-CD73 antibody. Combined therapy with immunotherapy against an inhibitory ICP and/or with a chemotherapeutic agent sensitive to MDR1 exclusion activity is proposed.

The present invention is related to CD73 expression by certain cells in tumoral process and to determination of the presence of such cells in a patient, allowing to adapt anti-cancerous therapy to said patient.

### BACKGROUND OF THE INVENTION

CD73 (ecto-5′-nucleotidase) is a 70-kDa glycosylphosphatidylinositol (GPI)-anchored protein normally expressed on endothelial cells and subsets of hematopoietic cells. CD73, together with CD39, regulates adenosine triphosphate (ATP) metabolism. CD39 (NTPDase-1) converts ATP into AMP, with only trace amounts of ADP being released, while CD73 catalyzes the conversion of AMP to adenosine (Ado).

Extracellular Ado accumulates in cancerous tissues and constitutes an important mechanism of tumor immune escape. Among other effects, tumor-derived Ado profoundly inhibits infiltrating effector T cells. ATP degradation into Ado through CD39 and CD73 co-expressed on murine Treg (regulatory CD4+ T cells) has been shown as responsible for tumor immunosuppression.

CD73 expression has been reported in a range of tumor cells, and therapy with an antibody that binds CD73 has been proposed.

### SUMMARY OF THE INVENTION

The inventors have demonstrated that in contrast to murine Treg, human Treg only express CD39 and thus were not able to generate Ado. Then the inventors characterized a subpopulation of memory CD4+ T cells depleted in Treg (CD4+ Tconv) that expressed CD73. They highlighted that CD73+CD4+ Tconv presented pronounced inflammatory Th1/17 capacities and these cells seems to be less sensitive to immuno-checkpoint (ICP) regulation. They are transformed to Th17 cells upon cooperation with CD39+ Treg through Ado generated in this cooperation and Th17 cells are protumoral.

More precisely, CD73 expression defines a discrete CD4+ T cell population of effectors with potent Th1/17 effector function (high IFNg, IL17, GM-CSF and IL22 production). CD73+CD4+ T cells are selectively inhibited by CD39+ Treg through the cooperative production of potent immunosuppressant Ado. In models with distance between cells only CD73+CD4+ T cells are inhibited through an autocrine Ado production. In the tumor environment Treg percentages are increased and they expressed upregulated levels of CD39 leading to complete inhibition of CD73+CD4+ T cell subset, through Ado production. Generated Ado inhibits all cytokines produced by CD73+CD4+ T cells except IL17, transforming a potent anti-tumor effector (through IFNg, IL22, IL17, IL2, GM-CSF) into an inflammatory pro-tumoral cell producing IL17 only. Majority of CD73+CD4+ T effectors are devoid in other negative ICP (PD1, CTLA4, Lag3, TIM3, TIGIT), and so far are only sensitive to Ado-mediated inhibition. Furthermore, upon in vitro TCR-mediated activation inhibitory ICP receptors strongly upregulated on CD73negCD4+ T cells, are only marginally induced in CD73+CD4+ T cells. CD73+CD4+ T effectors, in absence of exogenous ATP, are not significantly inhibited by monocytes in contrast to CD73negCD4+ T cells that are strongly inhibited. In the opposite, in presence of ATP, monocytes expressing CD39, like Treg, will completely block CD73+CD4+ T cell proliferation. Furthermore, this CD73+CD4+ population uniquely expresses high levels of the efflux channel MDR1 also known as multidrug resistance receptor.

Analysis of CD73 expression by IHC allows to evidence highly variable expression from tumor to tumor. Importantly, according to tumor CD73 expression can be completely undetectable or expressed at variable levels on tumor cells, endothelial cells, mesenchymal stromal cells and lymphocyte infiltrate, or any combinations. Finally, in melanoma patients treated by anti-PD1, no modulation of the frequency of the CD73+CD4+ population in the periphery was noticed upon anti-PD1 treatment.

All these data let the inventors to the following conclusions:

The CD73+CD4+ T cell subset is a potent anti-tumor effector population that in the tumor environment is inhibited through CD39/CD73 because of its CD73 expression, and will not be sensitive to other anti-inhibitory ICP as their expression is absent.

Thus the patient presenting a high frequency of this CD73+CD4+ T cells in the blood and/or their tumor, in particular when combined with high frequency of CD39+ cells (Treg, macrophages, MDSC, . . . ) might not respond to other immunotherapy, but should respond to anti-CD73 therapy, or combination therapy.

Because Ado is highly labile the expression of CD73 on the tumor might not lead to impaired T cell function because of distance

Thus the analysis of CD73 expression in tumor on immune cells infiltrate will represent a biomarker of resistance to other immunotherapies and to response to anti-CD73 therapy, or combination therapy.

Finally, the selective expression of MDR1 on this CD73+CD4+ T cell population represents a strong argument for combination therapy with chemotherapy, in particular in neo-adjuvant setting as MDR1⁺CD73⁺CD4⁺ Tconv are resistant to MDR-1 substrate chemotherapy (Taxol as an example) in contrast to MDR1^{neg} cells.

All these data led the inventors to conclude that expression of CD73 on tumor cells and endothelial cells is not a valid biomarker of whether or not an anti-CD73 therapy will be efficient or not, only CD73 expression on immune infiltrate and/or stromal cells contacting immune cells is a valid biomarker.

These data let also the inventors to propose CD73 expression on immune cells, such as immune infiltrate and/or stromal cells contacting immune cells or immune cells in the blood, as a biomarker of resistance to anti-PD1 and other conventional anti-inhibitory ICP immunotherapies, and of combination therapy with anti-CD73 antagonist drugs.

An object of the invention is a method for selecting a human patient for CD73 antagonist therapy, preferably immunotherapy, comprising the step of determination in a patient's biological sample, of lymphocytes, preferably CD4+ T cells, expressing CD73, especially at elevated frequency or higher frequency than a healthy patient or healthy solid tissue. The method may also comprise determining presence of Treg and/or monocytes expressing CD39. The method may be immunohistochemistry (IHC) or immunofluorescence (IF), single or multiple.

In an embodiment, the biological sample is tumor tissue and/or its environment. Preferably, the method comprises determining infiltrating lymphocytes or stromal elements contacting lymphocytes, preferably CD4+ T cells, in the tumor environment expressing CD73. The method may also comprise determining presence of Treg and/or monocytes expressing CD39 in the same sample.

Upon a determination that infiltrating lymphocytes or stromal elements contacting lymphocytes, preferably CD4+ T cells, in the tumor environment express CD73 in patient's biological sample, especially at a given level or frequency, the patient is declared sensitive to immunotherapy using an antagonist of CD73, preferably an anti-CD73 antibody.

In an embodiment, the biological sample is blood or a blood derivative containing circulating cells. Preferably, the method comprises determining that lymphocytes, in the blood, express CD73. The method may also comprise determining presence of Treg and/or monocytes expressing CD39 in the same sample or in a sample of tumoral tissue or its environment. The method may use flow cytometry.

Upon a determination that lymphocytes, preferably CD4+ T cells, in blood express CD73 in patient's biological sample, especially at a given level or frequency, the patient is declared sensitive to immunotherapy using an antagonist of CD73, preferably an anti-CD73 antibody.

In an embodiment, determination is made in a patient's biological sample, that CD4+ T cells infiltrating the tumor environment or the tumor or CD4+ T cells in blood, express CD73 and, based on the finding that CD73 expressing CD4+ T cells also express MDR1, it is concluded that these cells will be sensitive to a combined therapy directed against CD73 on the one hand with an antagonist of CD73, preferably an anti-CD73 antibody, together with a chemotherapeutic agent sensitive to MDR1 exclusion activity (Taxol as an example). In an embodiment, MDR1 expression by the CD73 expressing CD4+ T cells is made. Determination of the expression of CD73 and MDR1 may be performed simultaneously.

In an embodiment, determination of the presence or frequency of CD39 expressing cells either in blood or in the tumor is performed.

Another object of the invention is a CD73 antagonist, preferably an anti-CD73 antibody, for use in treating a human patient against cancer, wherein the patient's tumor environment comprises CD73-expressing lymphocytes (infiltrating lymphocytes or stromal element contacting lymphocytes), and/or the blood comprises CD73-expressing lymphocytes, preferably CD4+ T cells. This status may have been previously assessed using the method of selection described herein. In an embodiment, CD39 expressing cells are present either in blood or in the tumor.

In an embodiment, the CD73 antagonist is for use in treating a patient against cancer in combination with a chemotherapeutic agent sensitive to MDR1 exclusion activity (Taxol as an example).

Another object of the invention is a method of treatment of a human patient against cancer, wherein the patient's tumor environment comprises CD73-expressing lymphocytes (infiltrating lymphocytes or stromal element contacting lymphocytes), and/or the blood comprises CD73-expressing lymphocytes, preferably CD4+ T cells, and the patient is administered an efficient amount of an antagonist of CD73, preferably an anti-CD73 antibody. This status may have been previously assessed using the method of selection described herein.

In an embodiment, the method of treatment comprises:
determining in the human patient if infiltrating lymphocytes or stromal element contacting lymphocytes, preferably CD4+ T cells, in the tumor environment and/or lymphocytes, preferably CD4+ T cells, in the blood, express CD73,
upon a determination that infiltrating lymphocytes or stromal element contacting lymphocytes, preferably CD4+ T cells, in the tumor environment or lymphocytes, preferably CD4+ T cells, in the blood, express CD73, administering to the patient an antagonist of CD73, preferably an anti-CD73 antibody.

In an embodiment, determination is made in a patient's biological sample, that infiltrating lymphocytes or stromal element contacting lymphocytes in the tumor environment, and/or the blood comprises CD73-expressing lymphocytes, preferably CD4+ T cells, express both CD73.

In an embodiment, the patient is also administered with an efficient amount of a chemotherapeutic agent sensitive to MDR1 exclusion activity (Taxol as an example). This embodiment may be performed on patient for which it has been determined the co-expression of MDR1 and CD73 by the CD4+ T cells.

In an embodiment, determination of the presence or frequency of CD39 expressing cells either in blood or in the tumor is performed.

Another object of the invention is a method for assessing in a human patient a risk of resistance to, or lack of efficacy of, cancer immunotherapy directed against an inhibitory ICP, comprising the step of determination in a patient's biological sample that tumor environment comprises CD73-expressing lymphocytes (infiltrating lymphocytes or stromal element contacting lymphocytes), and/or the blood comprises CD73-expressing lymphocytes, wherein detecting CD73-expressing lymphocytes is indicative of a risk of resistance to, or lack of efficacy of, cancer immunotherapy directed against said inhibitory ICP. CD73-expressing cells may be human CD73-expressing lymphocytes, or more specifically CD4+ T cells expressing CD73. In an embodiment, said lymphocytes, preferably CD4+ T cells, express both CD73 and MDR1. In an embodiment, determination of the presence or frequency of CD39 expressing cells either in blood or in the tumor is performed.

The invention encompasses any usual immunotherapy against an inhibitory ICP implicated in tumor immunosuppression. Typical ICP is PD1, CTLA4, Lag3, TIM3, TIGIT.

In an embodiment the ICP is PD1 (or PD-1) and immunotherapy inhibits the PD1 axis, i.e. inhibits PD1 or PD-L1.

In an embodiment the ICP is CTLA4 and immunotherapy inhibits the CTLA4 axis.

In an embodiment, the patient is also administered with an efficient amount of a chemotherapeutic agent sensitive to MDR1 exclusion activity.

Upon a determination that infiltrating lymphocytes or stromal element contacting lymphocytes, preferably CD4+ T cells, in the tumor environment express CD73 in patient's biological sample, and/or the blood comprises CD73-expressing lymphocytes, the patient is declared at risk of being resistant or of lack of response or efficacy to immunotherapy against an inhibitory ICP.

In an embodiment, it is considered that the patient may benefit from immunotherapy using an antagonist of CD73, preferably an anti-CD73 antibody combined with immunotherapy against an inhibitory ICP, such as PD1 of CTLA4. In this embodiment, decision may be made to treat the patient with an antagonist of CD73, preferably an anti-CD73 antibody, in addition to the immunotherapy against said inhibitory ICP.

Another object of the invention is a CD73 antagonist for use in treating a patient against cancer, wherein the patient is to be treated or is being treated with a cancer immunotherapy directed against an inhibitory ICP and the tumor environment in said patient comprises CD73-expressing lymphocytes (infiltrating lymphocytes or stromal element contacting lymphocytes) and/or the blood comprises CD73-expressing lymphocytes. The CD73-expressing lymphocytes may be in particular CD73-expressing CD4+ T cells. In an embodiment, CD39 expressing cells are present either in blood or in the tumor.

In an embodiment, the antagonist is an antibody directed against CD73, for example MEDI9447 or 7G2.

In an embodiment, the patient is to be treated or is being treated with a cancer immunotherapy directed against an inhibitory ICP which is PD1, CTLA4, Lag3, TIM3, TIGIT, preferably PD1 or CTLA4.

In an embodiment, the patient is also administered with an efficient amount of a chemotherapeutic agent sensitive to MDR1 exclusion activity (Taxol as an example). This embodiment may be performed on patient for which it has been determined the co-expression of MDR1 and CD73 by the CD4+ T cells.

Another object of the invention is a method of treatment of a human patient against cancer, wherein the patient's tumor environment comprises CD73-expressing lymphocytes (infiltrating lymphocytes or stromal element contacting lymphocytes), and/or the blood comprises CD73-expressing lymphocytes, preferably CD4+ T cells, and the patient is treated by immunotherapy directed against an inhibitory ICP, combined with treating the patient with an antagonist of CD73, preferably an anti-CD73 antibody. This status may have been previously assessed using the method of selection described herein. In an embodiment, said cells also express MDR1. In an embodiment, CD39 expressing cells are present either in blood or in the tumor.

In an embodiment, the method of treatment comprises:
determination that tumor environment comprises CD73-expressing lymphocytes (infiltrating lymphocytes or stromal element contacting lymphocytes), and/or the blood comprises CD73-expressing lymphocytes, wherein detecting CD73-expressing lymphocytes is indicative of a risk of resistance to, or lack of efficacy of, cancer immunotherapy directed against an inhibitory ICP, and
upon determination that tumor environment and/or blood comprises CD73-expressing lymphocytes, treating the patient with cancer immunotherapy directed against said inhibitory ICP, combined with treating the patient with an efficient amount of an antagonist of CD73, preferably an anti-CD73 antibody.

In an embodiment, the patient is also administered with an efficient amount of a chemotherapeutic agent sensitive to MDR1 exclusion activity. This embodiment may be performed on patient for which it has been determined the co-expression of MDR1 and CD73 by the CD4+ T cells.

In an embodiment, determination of the presence or frequency of CD39 expressing cells either in blood or in the tumor is performed.

Other objects will appear in the detailed description.

### DETAILED DESCRIPTION

1.0 The invention thus provides for a method for selecting a human patient for CD73 antagonist therapy, preferably immunotherapy, comprising the step of determination in a patient's (available, i.e. previously obtained) biological sample, that lymphocytes, preferably CD4+ T cells, express CD73, wherein, upon a determination that lymphocytes, preferably CD4+ T cells, express CD73 in patient's biological sample, the patient is declared sensitive to immunotherapy using an antagonist of CD73, preferably an anti-CD73 antibody, or decision is made to treat said patient using an antagonist of CD73, preferably an anti-CD73 antibody.

This method may comprise the determination (i) in a tumoral tissue or environment sample whether infiltrating lymphocytes or stromal element contacting lymphocytes, preferably CD4+ T cells, and/or (ii) determination in blood sample whether lymphocytes, preferably CD4+ T cells, express CD73.

This determination may be associated with determination of the presence of immune cells, in particular Treg and/or monocytes, expressing CD39, for example in the tumor or its environment, such as infiltrating Treg and/or monocytes expressing CD39.

In an embodiment, MDR1 expression by the CD73 expressing CD4+ T cells is made. Determination of the expression of CD73 and MDR1 may be performed simultaneously.

Upon said determination of cells expressing CD73, possibly MDR1 as well, and possibly of cells expressing CD39 as well, the patient is declared sensitive to combined therapy with an antagonist of CD73, preferably an anti-CD73 antibody, and chemotherapy using a chemotherapeutic agent sensitive to MDR1 exclusion activity, or decision is made to treat said patient with such combined therapy.

In an embodiment, determination that lymphocytes, preferably CD4+ T cells, express CD73 in patient's biological sample, and possibly presence of immune cells, in particular Treg and/or monocytes, expressing CD39, allows to conclude that the human patient is at risk of resistance to, or lack of efficacy of, cancer immunotherapy directed against an inhibitory immune checkpoint (ICP).

The invention also provides a CD73 antagonist, preferably an anti-CD73 antibody, for use in treating a human patient against cancer, wherein the patient comprises CD73-expressing lymphocytes, preferably CD4+ T cells. Preferably, said human patient has tumour infiltrating lymphocytes or stromal element contacting lymphocytes, preferably CD4+ T cells, and/or blood lymphocytes, preferably CD4+ T cells, expressing CD73. Still preferably, said patient further has immune cells, in particular Treg and/or monocytes, expressing CD39.

In an embodiment, said antagonist is for use in a combined therapy with (i) a chemotherapeutic agent sensitive to MDR1 exclusion activity, (ii) cancer immunotherapy directed against an inhibitory ICP, or (iii) both.

The invention also provides a method for assessing in a human patient a risk of resistance to, or lack of efficacy of, cancer immunotherapy directed against an inhibitory immune check point (ICP), comprising the step of determination in a patient's biological sample whether lymphocytes, more specifically CD4+ T cells, expressing CD73 are present, wherein detecting CD73-expressing lymphocytes is indicative of a risk of resistance to, or lack of efficacy of, cancer immunotherapy directed against said inhibitory ICP. In a non-limiting embodiment, as it will appear from the foregoing, said inhibitory ICP immunotherapy is an anti-PD1 or anti-CTLA4 immunotherapy.

In an embodiment, said method further comprises determination of the presence of immune cells, in particular Treg and/or monocytes, expressing CD39.

In an embodiment, upon a detection of CD73-expressing lymphocytes, and possibly cells expressing CD39, the patient is declared sensitive to treatment with an antagonist of CD73, preferably an anti-CD73 antibody, in addition to the immunotherapy against said inhibitory ICP, or decision is made of treating said patient with an antagonist of CD73, preferably an anti-CD73 antibody, in addition to the immunotherapy against said inhibitory ICP.

In an embodiment, the patient is declared sensitive to treatment with a chemotherapeutic agent sensitive to MDR1 exclusion activity, or decision is made to treat the patient with such agent in addition to the treatment with an antagonist of CD73, preferably an anti-CD73 antibody, and the immunotherapy against said inhibitory ICP.

The invention also provides for a CD73 antagonist, preferably anti-CD73 antibody, for use in treating a patient against cancer, wherein the patient is to be treated or is being treated with an immunotherapeutic agent, such as antibody, directed against an inhibitory ICP, preferably anti-PD1 or anti-CTLA4 and/or a chemotherapy with a chemotherapeutic agent sensitive to MDR1 exclusion activity, and said patient comprises CD73-expressing lymphocytes, preferably CD4+ T cells.

The invention also provides for a kit comprising a CD73 antagonist, preferably anti-CD73 antibody, and a chemotherapeutic agent sensitive to MDR1 exclusion activity, for simultaneous, separate or sequential use to treat cancer in a patient comprising CD73-expressing lymphocytes, preferably CD4+ T cells.

The invention also provides for a kit comprising a CD73 antagonist, preferably anti-CD73 antibody, and an immunotherapeutic agent, such as antibody, directed against an inhibitory ICP, preferably anti-PD1 or anti-CTLA4, for simultaneous, separate or sequential use to treat cancer in a patient comprising CD73-expressing lymphocytes, preferably CD4+ T cells.

The kit may also comprise the combination of the three kind of agent described above.

The invention also provides a method of treatment of a human patient against cancer, wherein the patient's tumor environment comprises CD73-expressing lymphocytes (infiltrating lymphocytes or stromal element contacting lymphocytes), and/or the blood comprises CD73-expressing lymphocytes, preferably CD4+ T cells, and the patient is treated with an efficient amount of a chemotherapeutic agent sensitive to MDR1 exclusion activity and/or with an efficient amount of an immunotherapeutic agent, such as antibody, directed against an inhibitory ICP, preferably anti-PD1 or anti-CTLA4, combined with treating the patient with an efficient amount of an antagonist of CD73, preferably an anti-CD73 antibody. This status may have been previously assessed using the method of selection described herein. In an embodiment, CD39 expressing cells are present either in blood or in the tumor.

Determination of CD73 expression may be performed on a patient's biological sample of a tissue selected from the group consisting of cancer tissue, tissue proximal to or at the periphery of a cancer, cancer adjacent tissue, adjacent non-tumorous tissue or normal adjacent tissue.

Determination may be done by immunohistochemistry (IHC) or immunofluorescence (IF) staining, using anti-CD73 and/or anti-CD4 antibodies.

Determination may involve simple staining by IHC coupled to pathologist scoring allowing identification of expression on tumor cells, endothelial cell, mesenchymal stromal cells or lymphocytes. Determination may also involve double IHC or double IF stainings CD4/CD73, in order to detect or get the level of CD4+ T cells expressing CD73.

Tumor or tumor environment tissue samples, e.g. micro-array (TMA), may be used and submitted to staining with an anti-hCD73 monoclonal antibody, e.g. anti-hCD73 rabbit monoclonal antibody (e.g. Clone D7F9A, Cell Signaling) followed by an amplification step with a secondary antibody, e.g. anti-rabbit Ab (e.g. kit OmniMap anti-Rb HRP, Roche) and the staining may be revealed with an appriopriate label or chromogen, e.g. an HRP chromogene (e.g. DAB) (kit ChromoMap DAB, Roche). Cells included in the assay may be, for example, tumoral cells, vessels, lymphocytes infiltrating the tumor and/or stromal cells.

In an embodiment, the biological sample is blood or a blood derivative containing circulating cells and flow cytometry may be used.

Cells may be stained with proliferation markers such as, for example, CTV and CFSE, and cultured in presence of expand beads such as, for example, anti-CD3/anti-CD28 beads (e.g. expand beads, 1 bead: 4 cells ratio) in plates such as, for example, 96 well flat-bottomed plates. Proliferation may be assessed by the respective analysis of the marker (e.g. CTV and CFSE) cell tracer dilution by flow cytometry.

Possible MDR1 cell expression may be measured by various known methods including cytometry on blood or tumor biopsies, or immunofluorescence. One may cite multiparametric cytometry on peripheral blood or on tumor biopsies, as per the method illustrated with FIG. 5. One may also use multiplex immunofluorescence allowing the determination of MDR1, CD73 and CD4, through revelation using antibodies to MDR1 such as E1Y7B (Rabbit Ab), to CD73 such as D7F9A (rabbit Ab) and to CD4 such as Clone SP35 (murine IgG1).

Example procedure for CD73/MDR1 co-expression on CD4+ T cells on Frozen tissue sections (example with primary breast tumor, applicable to other tumors): Tissue-Tek® O.C.T. (Sakura® Finetek) embedded frozen human primary breast tumors are used to generate 6 μm frozen tissue sections with Cryotome™ (Thermo Fisher Scientific). Sections are fixed in paraformaldehyde, permeabilized in Triton X-100 and stained. Slides are saturated with PBS1×5% mouse and donkey serum (containing triton 0.01%) for one hour. The sections are then incubated with the primary antibodies coupled to specific fluorochromes such as MDR1/CD243 coupled to PE (clone UIC-2), CD4 coupled to AF488 (clone SP35) and uncoupled CD73 (monoclonal rabbit D7F9A) for 2 hours. To reveal CD73, sections are then incubated with the donkey anti-rabbit secondary antibody (coupled to APC) for 1 hour at room temperature. After staining nuclei with DAPI, slides are mounted in ProLong Gold® mounting medium. Once dry, slides are conserved at 4° C. and Immunofluorescence stainings are analyzed on Upright Microscope (Nikon Ni-E) using ImageJ free software.

As antagonist of CD73, one may use preferably small molecules or antibodies. Any known efficient anti-CD73 antibody may be used, for example antibody MED19447 (J C Geoghegan et al., MAbs. 2016 Feb. 8:1-14) or antibody 7G2 (Sabastian F M Hausler et al., Am J Transl Res. 2014; 6(2):129-139).

There are currently at least five agents blocking the PD1/PD-L1 pathway that are marketed or in clinical evaluation, any of these may be useful in combination with the anti-CD73 therapy in the context of the invention. These agents are BMS-936558 (anti-PD-L1 mAb, Nivolumab/ONO-4538, Bristol-Myers Squibb, formerly MDX-1106—antibody 5C4 in WO 2006/121168), MK-3475 (anti-PD1 mAb, lambrolizumab or pembrolizumab, Keytruda®, Merck), MPDL3280A/RG7446 (anti-PD-L1 mAb, Roche/Genentech), AMP-224 (immunoadhesin comprising an anti-PD-L2, Amplimmune and GSK), Pidlizumab (anti-PD1 mAb, CT-011, CureTech/TEVA—WO 2009/101611).

For MK-3475 DNA constructs encoding the variable regions of the heavy and light chains of the humanized antibodies h409All have been deposited with the American Type Culture Collection Patent Depository (10801 University Bld., Manassas, Va.). The plasmid containing the DNA encoding the heavy chain of h409A-I 1 was deposited on Jun. 9, 2008, and identified as 081469_SPD-H and the plasmid containing the DNA encoding the light chain of h409AI 1 was deposited on Jun. 9, 2008 and identified as 0801470_SPD-L-I 1.

Further known PD-1 antibodies and other PD-1 inhibitors include AMP-224 (a B7-DC/IgG1 fusion protein licensed to GSK), AMP-514 described in WO 2012/145493, antibody MEDI-4736 (an anti-PD-L-1 developed by AstraZeneca/Medimmune) described in WO2011/066389 and US2013/034559, antibody YW243.55.570 (an anti-PD-L1) described in WO2010/077634, MDX-1105, also known as BMS-936559, is an anti-PD-L1 antibody developed by Bristol-Myers Squibb described in WO2007/005874, and antibodies and inhibitors described in WO2006/121168, WO2009/014708, WO2009/114335 and WO2013/019906. The disclosures of any document referred to herein are hereby incorporated by reference. Further examples of anti-PD1 antibodies are disclosed in WO2015/085847 for examples antibodies having light chain variable domain CDR1, 2 and 3 of SEQ ID NO:6, SEQ ID NO: 7 and SEQ ID NO: 8, respectively, and antibody heavy chain variable domain CDR1, 2 and 3 of SEQ ID NO: 3, SEQ ID NO: 4 and SEQ ID NO: 5, respectively, wherein the SEQ ID NO references are the numbering according to WO2015/085847.

CTLA-4 (cytotoxic T-lymphocyte-associated protein 4), also known as CD152 is another inhibitor member of the CD28 family of receptors, and is expressed on T cells. Antibodies that bind and inhibit CTLA-4 are known in the art and may be used herein in combination with the anti-CD73. In one example, the antibody is ipilimumab (trade name Yervoy®, Bristol-Myers Squibb), a human IgG antibody.

Chemotherapeutic agents sensitive to MDR1 exclusion are known and may be selected from the group consisting of Taxol, Daunorubicin, Docetaxel, Doxorubicin, Etoposide, Imatinib (imatinib mesylate), Irinotecan, Melphalan, Mitoxantrone, Paclitaxel, Sirolimus, Tacrolimus, Teniposide, Topotecan, Valspodar, Verapamil, Vinblastine, Vincristine, actinomycin, irinotecan (CPT-11), methotrexate, cytarabine, 5-fluorouracil, hydroxyurea, paclitaxel, docetaxel, chlorambucil, cisplatin, gefitinib, tamoxifen and bisantrene.

In the context of the invention, the term “treating” or “treatment”, as used herein, means reversing, alleviating, inhibiting the progress of, or preventing the disorder or condition to which such term applies, or one or more symptoms of such disorder or condition.

By the term “treating cancer” as used herein is meant the inhibition of the growth of malignant cells of a tumour and/or the progression of metastases from said tumor. Such treatment can also lead to the regression of tumor growth, i.e., the decrease in size of a measurable tumor. In a particular embodiment, such treatment leads to a partial regression of the tumor or metastasis. In another particular embodiment, such treatment leads to the complete regression of the tumor or metastasis. In some aspect, treatment prevents metastasis.

According to the invention, the term “patient” or “patient in need thereof” is intended for a human or non-human mammal affected or likely to be affected with a malignant tumor.

By a “efficient amount” is meant a sufficient amount of the active agents to treat said cancer disease, at a reasonable benefit/risk ratio applicable to any medical treatment. It will be understood, however, that the total daily usage of the active agents will be decided by the attending physician within the scope of sound medical judgment. The specific therapeutically effective dose level for any particular patient will depend upon a variety of factors including the disorder being treated and the severity of the disorder; activity of the specific active principle, e.g. antibody employed; the specific composition employed, the age, body weight, general health, sex and diet of the patient; the time of administration, route of administration, and rate of excretion of the specific active agents employed; the duration of the treatment; drugs used in combination or coincidental with the specific active agents employed; and like factors well known in the medical arts. In a further embodiment, the active agents of the invention is administered repeatedly according to a protocol that depends on the patient to be treated (age, weight, treatment history, etc.), which can be determined by a skilled physician.

The active principles are formulated with pharmaceutically acceptable carriers, vehicles or excipients. “Pharmaceutically” or “pharmaceutically acceptable” refers to molecular entities and compositions that do not produce an adverse, allergic or other untoward reaction when administered to a mammal, especially a human, as appropriate. A pharmaceutically acceptable carrier, vehicle or excipient refers to a non-toxic solid, semi-solid or liquid filler, diluent, encapsulating material or formulation auxiliary of any type.

The form of the pharmaceutical compositions including the active principles; e.g. antibody of the invention and the route of administration naturally depend upon the condition to be treated, the severity of the illness, the age, weight, and gender of the patient, etc.

The active agents of the invention can be formulated for a topical, oral, parenteral, intranasal, intravenous, intramuscular, subcutaneous or intraocular administration and the like. In a particular embodiment, the active agents of the invention are administered intravenously.

In particular, the pharmaceutical compositions including the active agents of the invention may contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

To prepare pharmaceutical compositions, an effective amount of the active agents of the invention may be dissolved or dispersed in a pharmaceutically acceptable carrier or aqueous medium.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi.

The carrier can be a solvent or dispersion medium containing, for example, water, ethanol, polyol (for example, glycerol, propylene glycol, and liquid polyethylene glycol, and the like) and suitable mixtures thereof. The proper fluidity can be maintained, for example, by the use of a coating, such as lecithin, by the maintenance of the required particle size in the case of dispersion and by the use of surfactants, stabilizing agents, cryoprotective or antioxidants. The prevention of the action of microorganisms can be brought about by antibacterial and antifungal agents. In many cases, it will be preferable to include isotonic agents, for example, sugars or sodium chloride.

Sterile injectable solutions are prepared by incorporating the active agents in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 mL of isotonic NaCl solution and either added to 1000 mL of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject.

As used herein, “simultaneous” is used to mean that the two agents are administered concurrently, whereas the term “sequential” is used to mean they are administered within a timeframe that they both are available to act therapeutically within the same time-frame. Thus, administration “sequential” may permit one agent to be administered within 5 minutes, 10 minutes or a matter of hours after the other provided the circulatory half-life of the first administered agent is such that they are both concurrently present in therapeutically effective amounts. The time delay between administrations of the components will vary depending on the exact nature of the components, the interaction there between, and/or their respective half-lives. In contrast, “separate” is used herein to mean that the gap between administering one agent and the other is significant i.e. the first administered agent may no longer be present in the bloodstream in a therapeutically effective amount when the second agent is administered.

The invention will now be described using non-limiting examples referring to the figures.

### FIGURES

FIG. 1: CD73, not co-expressed with CD39 on Treg, is detected on memory CD4⁺ Tconv (CD39 and CD73 are distinctly expressed on human T cells). (A) Representative expression of CD39 and CD73 analyzed on blood HD memory CD4⁺ Treg by flow cytometry. (B) Cumulative data from twelve individual donors. (C) Representative expression of CD39 and CD73 analyzed on HD blood memory Tconv by flow cytometry. Data are representative of the twelve individuals donors tested.

FIG. 2: According to their localization, CD73⁺ Tconv are preferential target or cooperate with Treg to induce inhibition through generation of Ado. The impact of ATP (75 μM/day) and Treg alone or in combination on the proliferation on purified CD73⁺ or CD73^{neg} Tconv at a ratio 1:1 was performed in Transwell plates. CD73⁺ and CD73^{neg} memory CD4⁺ Tconv were respectively cultured in the upper and lower chamber in presence of anti-CD3/anti-CD28 beads at ratio beads to cells of 1:4 with or without ATP (75 μM) (1) and Treg were added either in the upper (2) or lower chamber (3).

FIG. 3: Breast and ovarian tumors contain highly functional CD39+ Treg and CD73+ Tconv with Th1/17 characteristics. (A) Representative expression of CD39 and CD73 analyzed on memory CD4+ Treg and Tconv from Breast (B) and ovarian (OT) by flow cytometry. Frequency of CD39+ cells (B) and Δ CD39 MFI on these cells (C) among paired Treg and Tconv were analyzed by flow cytometry among BT and OT and statistical analysis was performed by Two-way Anova analysis. (D) Purified tumor-associated Treg were assessed for ATP (37.5 μM) degradation by HPLC measurements and compared to HD-blood Treg. In some conditions 30 min pre-incubation with ARL67156 (250 μM) was used to assess the role of CD39 functional activity. (E,F) Δ CD39 MFI of CD39+ cells among Treg (E) or Tconv (F) from paired peripheral blood and BT or OT were analyzed. Statistical analysis was performed by using Two-way Anova analysis. (G) Frequency of CD73+ cells among Tconv from BT and OT was analyzed by flow cytometry. Statistical analysis was performed by using Kruskal-Wallis test.

FIG. 4: CD73⁺ Tconv express less Immuno-checkpoint (A) TIGIT and CTLA-4 gene expression were extracted from transcriptomic analysis of CD73⁺ or CD73^{neg} Tconv before and after short term activation with anti-CD3/anti-CD28 beads. Statistical analyses were performed using Two-way Anova analysis. (B) Representative expression of inhibitory ICPs (TIGIT, CTLA-4, TIM-3, PD-1) and CD73 analyzed by flow cytometry on. Tconv from HD-blood (HD-Tconv) or on Tumor-inflitrating Tconv (Ti-Tconv).

FIG. 5: CD73⁺Tconv present phenotypic and functional characteristics of Th1/17 cells. (A) MDR1 expression was assessed by flow cytometry using an anti-human MDR1 antibody (UIC2 clone) for 20 min at 37° C. in the presence of Cyclosporin A (25 μM, R&D Systems) together with CD73 (AD2 clone) on Tconv. (B, C) MDR1 functionality was assessed on CD73⁺ and CD73^{neg} Tconv by their capacity to exclude Rhd123 (1 μg/ml) analyzed by flow cytometry. The specificity was confirmed using Elacridar (1 μM), a MDR1 specific inhibitor. Frequency of Rhd123^{neg} cells was analyzed among CD73⁺ (black) and CD73^{neg} (white) Tconv (D). Statistical analysis was performed by using Wilcoxon test.

FIG. 6: In presence of exogenous ATP, CD39+ monocytes block proliferation of both CD73+ and CD73neg subsets, but only CD73+ depends on CD39/CD73. HD blood cell-sorted CD73⁺ and CD73^{neg} memory CD4⁺ Tconv were stained respectively with CTV and CFSE proliferation markers and cultured at a concentration of 3.10⁴/200 μl for 4 days in presence of anti-CD3/anti-CD28 beads (expand beads, 1 bead: 4 cells ratio) in 96 well flat-bottomed plates. Proliferation was assessed by the respective analysis of the CTV and CFSE cell tracer dilution by flow cytometry. The impact of ATP (75 μM/day) and monocytes (ratio 1:4) alone or in combination on the proliferation on CD73⁺ or CD73^{neg} Tconv was performed. The impact of CD73/CD39 axis was assessed by 30 min pre-incubation of cells with AMPCP (CD73 inhibitor, 50 μM) and ARL67156 (CD39 inhibitor, 250 μM) prior the addition of expand beads and/or ATP.

FIG. 7: CD73 expression in breast tumors. A retrospective FFPE-fixed breast tumor tissue micro-array (TMA) including all breast tumor molecular subtypes with associated clinical data base, has been subjected, after a process of antigen retrieval (Citrate buffer, pH7 for 30 min), and the degradation of endogenous peroxidase (with H₂0₂) to staining with an anti hCD73 rabbit monoclonal antibody (Clone D7F9A, Cell Signaling) at a dilution 1/200 for 60 min followed by an amplification step with a secondary anti rabbit Ab (kit OmniMap anti-Rb HRP, Roche) and the staining was revealed with an HRP chromogene (DAB)(kit ChromoMap DAB, Roche). All these processes were performed on a Ventana Discovery (Roche). In some tumors, tumoral cells are strongly stained by CD73 mAb (A) whereas in other tumors (B) only vessels (B), lymphocytes infiltrating breast tumors (C, D) or stromal cells (CAF=cancer associated fibroblasts) (D) express significant levels of CD73.

FIG. 8: CD73 is not significantly modulated on CD4+ T cells during anti-PD1 treatment in blood of metastatic melanoma patients. 2^{nd} line metastatic melanoma patients treated with anti PD-1 (Nivolumab, 3 mg/kg/i.v, every 2 weeks) were subjected to blood immune-monitoring, by multiparametric flow cytometry analysis, to assess, using specific coupled mAbs against CD73 (APC) and TIGIT (PE), the modulation of expression of both ICP on CD4+ and CD8+ T cells before (inclusion, WO) and during treatment (W2, W12). No significant modulation of CD73 was observed on the 11 patients evaluated either on CD4+ or on CD8+ T cells (A and B) whereas TIGIT expression was increased between WO and W2 on the representative dot plot presented (A).

FIG. 9: MDR-1 co-expression by CD73+ Tconv delineates a poly-functional population enriched in Th1/17 subset. 10⁶ PBMC were reactivated for 5 hours with PMA (50 ng/ml) and lonomycin (1 μg/ml) in the presence of Golgi-transport inhibitor (Brefeldin A, 10 μg/ml). At the end of the culture, cells were collected, washed, and stained with surface antibodies to look, using specific coupled antibodies, at CD73 and MDR1 expression within CD4⁺ memory T cells (CD3⁺CD4⁺CD45RA^{neg}) and quantify, after permeabilization, the production of IFNγ and TNFα (A) or IL-17 and IL-17/IFNγ coproduction (B) by the four subsets (CD73^{neg} MDR1 neg, CD73^{neg}MDR1⁺, CD73⁺MDR1^{neg}, CD73⁺MDR1⁺).

FIG. 10: In breast tumor environment, a majority of CD73⁺ Tconv is totally devoid in other inhibitory ICPs. The expression, on CD4⁺ memory Tconv (CD45⁺CD4⁺CD45R0⁺) of different inhibitory ICPs (TIGIT, CTLA-4, TIM-3, PD-1) was analyzed on 10 independent fresh primary breast tumors enzymatic disaggregation were analyzed by multiparametric flow-cytometry. Data were analyzed using the Boolean method on the FlowJo software the number of ICPs expressed (0 to 4) was then represented using SPICE v5.3 software according to either CD73 (A) or PD-1 (B) expression. This demonstrates that majority of CD73+ Tconv are totally devoid in other ICPs whereas most of PD-1+ Tconv also co-express other inhibitory ICPs.

FIG. 11: The expression of MDR1 renders CD73⁺CD4⁺ Tconv population resistant to apoptosis mediated by grading doses of Taxol, a recognized MDR1 substrate. Memory CD4⁺ Tconv purified from healthy donor PBMC were used. Purified 10⁵ CD4⁺ Tconv were cultured in 96-round bottomed wells in complete RPMI medium for 48 h in presence of a strong TCR stimulation (expand beads, ratio bead:cell 1:8)+IL-2 (50UI/ml) in absence (medium condition) or in presence of graded doses of Taxol (0.01; 0.1; 1 μM). Viability was analyzed by multiparametric flow cytometry on different cell subsets according to their expression of CD73 and MDR1 (CD73 MDR1, CD73⁺MDR1^{neg}, CD73^{neg}MDR1⁺, CD73^{neg}MDR1^{neg}). Survival was analyzed by multiparametric flow cytometry using Annexin-V and DAPI stainings (both from Becton Dickinson) according to CD73 and MDR1 expression. % of viable cells (Annexin V^{neg} DAPI^{neg}) was compared according to the cell subset analyzed. CD73 MDR1 cells are strongly resistant to Taxol effect whereas CD73^{neg} MDR1^{neg}are highly sensitive to Taxol treatment. Interestingly CD73^{neg} expressing MDR1 are also resistant to Taxol-induced apoptosis.

### EXAMPLES

CD39 and CD73 are Expressed by Distinct CD4⁺ T Cells with Memory Phenotype

In contrast to murine Treg co-expressing CD39 and CD73 we observed, by flow cytometry, that human healthy donor (HD) blood neither Treg nor total CD4⁺ T cells never co-expressed CD39 and CD73. Then we monitored CD39 and CD73 expression along the differentiation/maturation stage of HD-blood CD4⁺ according to their expression of CCR7, CD45RA, CD95, CD27 and CD28. Naïve CD4⁺ T cells did not express CD39. During CD4⁺ differentiation, CD39 expression peaked at the stage of memory effectors CD27+(CCR7^{neg}CD45RA^{neg)} (Mean±SEM: 18.29±9.67%). CD73 was almost not observed on naïve CD4⁺ T cells (Mean±SEM: 6.67±3.48%) but its expression significantly increased on central and effector memory CD4⁺ T cells (Mean±SEM: 11.02±6.67%).

In CD8 T cells, CD39 is also only found on memory cells whereas CD73 is mainly expressed on cells with naïve phenotype and decreased along differentiation but coexpression of both ectonucleotidase is never observed.

Among CD4⁺ T cells, we defined Treg based on their expression of the transcription factor forkhead box P3 Foxp3 or defined though the absence of expression of IL-7 Receptor (CD127) and the higher expression of IL-2 Receptor. Foxp3^{neg} CD4⁺ T cells or CD127+CD25^{neg/low} were determined as conventional T cells (Tconv). Based on CD45RO expression, the majority of CD4⁺ Treg exhibit memory phenotype in HD, patient's blood or in Breast and ovarian tumor. In contrast, half of CD4⁺ Tconv subsets are of memory phenotype in HD blood (Mean±SEM: 49.19±6.99%) and blood from breast cancer patients (BT-Blood) (Mean±SEM: 52.86±15.03%) or ovarian cancer patients (OT-Blood) (Mean±SEM: 50.11±11.52%) while CD4⁺ Tconv in tumor tissues are in majority of memory phenotype (Mean±SEM BT: 89.95±7.21%; OT: 82.91±10.38%). Thus we decided to focus our entire study on memory CD4⁺ T cells compartment.

Human Treg Express CD39 Whereas a Subset of Conventional CD4⁺ T Cells Express CD73

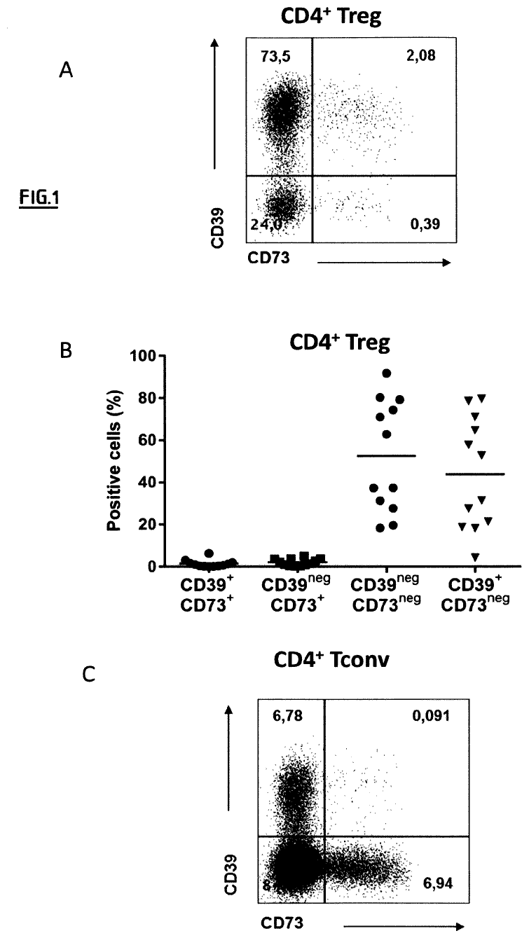

In contrast to murine Treg coexpressing CD39 and CD73, we showed in human that, HD Blood Treg expressed CD39 at variable levels but not CD73 (FIG. 1A, B). The absence of CD73 expression was also confirmed on purified blood Treg by western blot compared to a positive control (MDA-MB231 cell line) (not shown).

Among HD-Blood Tconv, we also detected a subset expressing CD39 but not CD73 (FIG. 1C) but at lower frequency and intensity than on Treg (ratio (delta CD39 MFI Treg/delta CD39 MFI Tconv) Mean±SEM: 1.64±0.33). Of interest, we identified a subset of Tconv expressing CD73 but not CD39 (mean±SEM: 5.43±2.24%) (FIG. 1C). Our results demonstrate that in contrast to murine Treg, human Treg only expressed CD39 and highlight the expression of CD73 on a subset of Tconv which may compensate the lack of CD73 on Treg to induce generation of Ado from ATP.

CD4⁺ CD73⁺ Tconv Present Characteristics of Th1/17 Cells (or Pathogenic/Inflammatory Th17 Cells)

CD73 expression on Tconv suggesting a role in Ado generation may denote a functional specialization of this population. Thus we analyzed CXCR3, CCR6 and CRTH2 expression to define Th1, Th17, Th2 and Th1/17 subset composition CD73⁺ Tconv showed similar frequency of Th1, Th17 and Th2 subsets compared to CD73^{neg} Tconv, but exhibited an enrichment in Th1/17 subset defined by the co-expression of CXCR3 and CCR6. Furthermore, in accordance to their phenotypic expression of CXCR3 and CCR6, CD73⁺ Tconv were more efficient than CD73^{neg} Tconv to migrate in response to CXCL10 and CCL20 in a Transwell migration assay (data not shown). Th17 cells are known to be found in mucosal tissues such as the intestine thanks to α4β7 integrin expression, thus we analyzed the presence of CD73⁺ Tconv in tonsil and colon tissue. We found a higher frequency of CD73⁺ Tconv in normal colon tissue but a lower frequency in tonsil tissue compared to HD-Blood. Then we analyzed by multiparametric flow cytometry intracellular stainings, the cytokine production pattern of CD73⁺ and CD73^{neg} Tconv from HD-Blood, single cell suspension of tonsil or colon tissues after PMA/ionomycin reactivation. Higher frequency of IFN-γ, TNF-α, IL-22 and IL-17A producing cells and more precisely IFN-γ/IL17A-co-producers were found within CD73⁺ Tconv (Mean±SEM: 1.69±0.71%) compared to CD73^{neg} from HD blood (Mean±SEM: 0.46±0.16%) and same observations were made in tissue.

To confirm this Th1/17 profile, CD73⁺ and CD73^{neg} Tconv subsets were sorted from HD-Blood memory CD4⁺ T cells by flow cytometry and following 24 hours PMA/ionomycin activation cytokine produced in supernatants were evaluated by Luminex. As observed through intracytoplasmic detection CD73⁺ Tconv secreted significantly more IFNγ, IL-17A, TNFα, IL-2, IL22 and also GM-CSF and IL-3 than their CD73^{neg} Tconv counterpart whereas they produced significantly lower levels of IL-10, IL-13 and IL-21.

In addition, we performed a transcriptomic analysis comparing purified HD-blood CD73⁺ and CD73^{neg} Tconv subsets before or after short term TCR stimulation (with anti-CD3/anti-CD28 beads). Interestingly genes associated with the Th1/17 also called pathogenic-Th17 signature defined by others groups were found overexpressed (csf2, abcb1, il22, il3, ifng, gzmb, il23r, ptger2 tbx21, lgals3) whereas specific Th17 genes were downregulated (lrmp, ikzf3, il10) in CD73⁺ Tconv subset compared to CD73^{neg} one.

The abcb1 gene, coding for multidrug transporter MDR1, was recently identified as a Th1/17 specific marker. The access, through gene expression omnibus (GEO) (accession ID: GSE49702), to the whole transcriptome of the MDR1⁺ and MDR1^{neg} CD4⁺ memory T cells performed in this study allowed us to observe that nt5e mRNA coding for CD73 is one of the most significantly increased gene in the MDR1⁺ population (Log 2-Fold Change=0.867; p value=0.004). These data are consistent with the significant higher expression of abcb1 gene in CD73⁺ Tconv in our microarray (Log-2 Fold Change=1.78; p value=0.010). To confirm MDR1 expression on CD73⁺ Tconv, we analyzed MDR1 on Tconv by flow cytometry and we observed that around half of CD73⁺ Tconv expressed this molecule compared to 20% in CD73^{neg} Tconv. Moreover, we took advantage of the physiological role of MDR1 in the efflux of specific fluorescent substrate like Rhodamine 123 (Rhd123). After staining of HD-PBMC with Rhd123 we analyzed, by flow cytometry, cells endowed with Rhd123 efflux function. CD73⁺ Tconv (HD-Blood mean 48.56±2.88) were able to mediate Rhd123 efflux compared to CD73^{neg} subset (HD-Blood mean 19.25±1.59) this being completely abrogated in the presence of Elacridar, a MDR1-specific inhibitor.

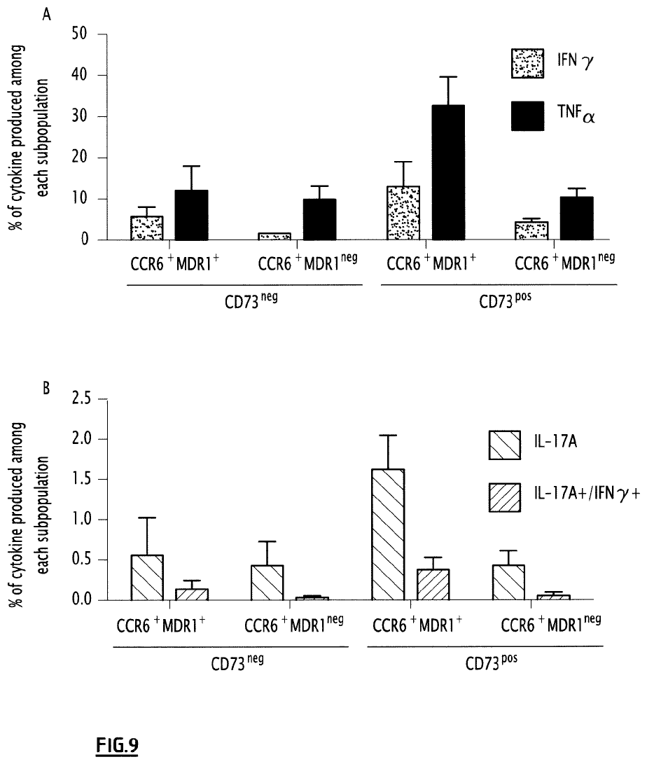

We investigated, on healthy donors PBMC, the impact of the addition of MDR1 staining (UIC-2 Ab combined with Cyclosporin-A for 30 min at 37° C.) together with CD73 in the multiparametric flow cytometry analysis to assess intracytoplasmic cytokine production capacity of CD4⁺ memory T cells in response to short term PMA/ionomycin reactivation (in the presence of Golgi-transport inhibitor). Whereas MDR1 did not discriminate the IFNγ, TNFα (FIG. 9A) or IL-17 production by CD73^{neg} Tconv (FIG. 9A), we demonstrated that within CD73⁺ Tconv population, MDR1 identified cells producing 3 fold more IFNγ, TNFα (FIG. 9A) and IL-17 (FIG. 9B) than the MDR1^{neg} counterpart. We confirmed also that CD73⁺ Tconv are enriched in Th1/17 (FIG. 9B) and the co-expression of MDR1 also segregated most (87%) of the Th1/17 cell subset within CD73⁺ Tconv. Taken together, these results confirmed that MDR1 allows to delineate, within CD73⁺ Tconv, highly polyfunctional population enriched in Th1/17 subset.

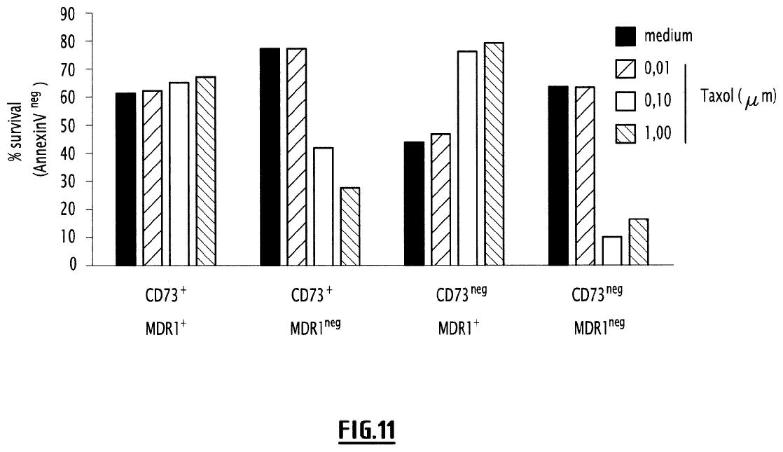

The selective resistance of CD73⁺MDR1⁺ Tconv population to chemotherapeutic drugs, substrate of MDR1, was evaluated in vitro in a 48 h assay using PBMC-purified memory CD4⁺ Tconv cultured at 10⁵cells/200 μl in complete RPMI medium in presence of a strong TCR stimulation with expand beads (ratio beads:cells 1:8)+IL-2 (50 IU/ml) in absence (medium condition) or in presence of graded doses of Taxol (0.01; 0.1; 1 μM). Their survival was analyzed by multiparametric flow cytometry using with Annexin-V and DAPI stainings (both from Becton Dickinson) according to CD73 and MDR1 expression. The percentage of viable cells (Annexin V^{neg} DAPI^{neg}) was compared according to the cell subset analyzed. As shown in FIG. 11, CD73⁺MDR1⁺ cells are strongly resistant to Taxol apoptosis as no modulation was observed after culture with Taxol compared to control medium. The CD73⁺MDR1^{neg} subset was more resistant than the CD73⁺MDR1⁺ to culture in presence of TCR stimulation alone (80% and 60% of viable cells respectively) but at high Taxol doses (0.1, 11 μM), this population was sensitive to apoptosis. The CD73^{neg}MDR1^{neg} population was found highly sensitive to Taxol treatment as only 16.6% of the cells remained viable at high doses of Taxol (0.1 and 1p M). Interestingly these results also demonstrated that CD73^{neg} cells expressing MDR1 that are spontaneously sensitive to apoptosis in medium alone (40% of viable cells after 48 h), were highly resistant to high Taxol concentrations (0.1, 1 μM) as 80% of viable cells were detected at 48 hours. Taken together these results demonstrate that MDR1 expression on CD73⁺ Tconv protect them from chemotherapeutic drugs toxicity and confirm the interest to use MDR1 substrates (Doxorubicin, Taxol, Vinblastine, etoposide) as chemotherapy in order to protect this polyfunctional population that will participate in the antitumor immune response.

Taken together, our results demonstrate that CD73 expression on Tconv highlight a subpopulation with multifunctional characteristics of Th1/17 cells that strongly resisted to Taxol-induced apoptosis in vitro through MDR1 expression.

Treg Cooperate with CD4⁺CD73⁺ Tconv to Degrade Extracellular ATP into Immunosuppressive Ado

The sole expression of CD39 by Treg suggests their ability to degrade ATP without generating Ado. To confirm this hypothesis, we quantified, by HPLC, the capacity of Treg to degrade ATP into Ado. To prevent interference with endogenous ATP or metabolites, these experiments were performed with exogenous C₁₃N₁₅ stable isotope ATP. After two hours, Treg purified from HD blood degraded 48.24±6.20% of ATP into AMP without generating neither Ado nor Inosine. The specific CD39 inhibitor (ARL67156) completely inhibited this degradation demonstrating the major role of CD39 ectonucleotidase enzymatic function. Moreover, we confirmed that CD73⁺ and CD73^{neg} Tconv were not able to degrade C₁₃N₁₅ ATP.

Using this technique, we confirmed the enzymatic functionality of CD73 on Tconv. Only CD73⁺ Tconv were capable of hydrolyzing C₁₃N₁₅ AMP into Ado and this was reversed by addition of a specific CD73 inhibitor (AMPCP).

Then, we analyzed the ability of Treg to cooperate with CD73⁺ Tconv to degrade ATP into Ado. Thus, Treg were co-incubated with CD73⁺ Tconv in presence of ATP leading to the generation of Ado but not Inosine. The addition of the combined ARL67156 and AMPCP inhibitors confirmed the involvement of CD39 and CD73 enzymatic functions.

Activated CD4⁺ Tconv Present an Ado-Sensitive Phenotype

As Ado exerts its immunosuppressive function by engaging A2a or A2b receptors, we analyzed our microarray data to assess A2aR (adora2a) and A2bR (adora2b) but also the enzyme Adenosine Deaminase ADA (ADA) converting Ado to Inosine at mRNA levels. We noticed that adora2a was expressed on resting CD73⁺ and CD73^{neg} Tconv and both adora2a and adora2b were increased after TCR-like stimulation, whereas adora1 (A1) and adora3 (A3) were not detected. In contrast, ada mRNA expressed at steady state was decreased after activation on both subsets. These data were also confirmed at protein level by Western Blot on purified subsets. Inversely to ADA that decreased upon activation, A2a and A2b receptors, not detectable at steady state, appeared after 1 day of stimulation and were highly increased after 4 days.

In 4 days' proliferation experiments with anti-CD3/anti-CD28 beads on purified CD73⁺ or CD73^{neg} Tconv, we demonstrated that the metronomic addition of exogenous Ado (75 μM/day, 300 μM final concentration) inhibited, the proliferation of both subsets as shown by the reduction of the size of the cell clusters compared to medium condition. Addition of recombinant ADA (rhADA, 1 μg/ml), inducing degradation of Ado into Ino restored both T cell subsets proliferation confirming the immunosuppressive impact of Ado. In contrast, the metronomic addition of AMP (150 μM final concentration) inhibited the proliferation of CD73⁺ Tconv but not CD73^{neg} Tconv, as a result of the ectonucleotidase function of CD73 as shown by the reversion of inhibition with the CD73 inhibitor AMPCP. These results were confirmed by flow cytometry analyzing the dilution of CFSE tracer on viable cells.

Moreover, the impact of Ado and AMP on cytokine secretion capacity was analyzed on both purified Tconv subsets. To avoid a confounding effect of Ado and AMP on proliferation in this analysis we analyzed cytokine secretion after 2 days of activation with anti-CD3/anti-CD28 beads which was determined as the maximal length of activation without induction of any proliferation (CFSE dilution). First, we confirmed that CD73⁺ Tconv produced more IL-2, TNF-α, IFN-γ, IL-17A, IL-22, GM-CSF and IL-3 but less IL-10, IL-13 and IL-21 than CD73^{neg} in this activation setting. As shown previously for the proliferation, the secretion of the cytokines produced by CD73⁺ or CD73^{neg} Tconv was inhibited by the addition of Ado. Interestingly, we observed that IL-17A secretion by Tconv CD73+(or Tconv CD73^{neg}) is not altered by the addition of Ado whereas IL-22 secretion was slightly inhibited.

Opposing to a Close Vicinity Environment, in a Spaced Out Environment, Treg Induce Ado-Mediated Inhibition of Only CD4⁺CD73⁺ Tconv without Affecting CD4⁺CD73^{neg} Tconv

To analyze the biological impact of Ado generation by cooperation between CD39⁺ Treg and CD73⁺ Tconv, we co-cultured Treg labeled with proliferation marker CFSE in presence of CD73⁺ or CD73^{neg} Tconv labeled with Cell Trace Violet (CTV) at a ratio 1/1 in presence or absence of exogenous ATP in round-bottomed wells with compact cell density in presence of anti-CD3/anti-CD28 beads. As previous observed by others, Treg did not present any sign of proliferation because of their anergic status (data not shown). In contrast, CTV-labeled CD73⁺ or CD73^{neg} proliferation that was not altered by the addition of Treg due to the strong activation. ATP alone did not induce inhibition of isolated Tconv subpopulations but the association of Treg and ATP induced strong inhibition of CD73⁺ Tconv, but not CD73^{neg} Tconv proliferation which is reversed by the addition of CD73 inhibitor (APCP). To mimic physiological conditions, Treg, CD73⁺ and CD73^{neg} Tconv subsets were mixed together at physiological ratio (10%/20%/70%) observed in blood. ATP induced inhibition of the bulk proliferation which can be rescued by the combined addition of CD39 and CD73 inhibitors. The proliferation of each subset was analyzed by specific proliferation markers (CFSE and CTV alone or in combination). Both CTV-labeled CD73⁺ Tconv and CFSE-labeled CD73^{neg} Tconv were inhibited in presence of ATP suggesting that Treg cooperated with CD73⁺ Tconv for ATP degradation into Ado that targeted CD73⁺ Tconv but also induced collateral inhibition of CD73^{neg} Tconv proliferation.

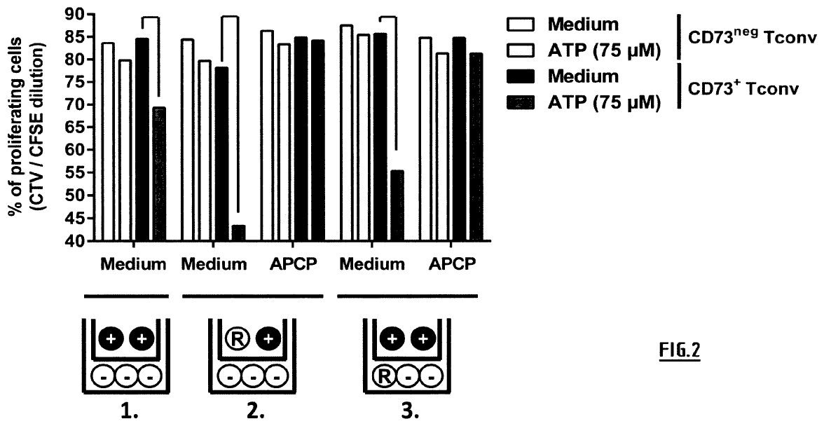

In the objective to assess whether CD73⁺ Tconv can be specifically targeted by Treg in a spaced out environment, we performed Transwell experiments in which CD73⁺ Tconv were cultured in the upper chamber and CD73^{neg} Tconv in lower one whereas Treg were added in one or the other according to conditions. We observed that alone or together in the Transwell, CD73⁺ and CD73^{neg} Tconv proliferated similarly than in previous experiments and addition of ATP had no negative impact. While the addition of Treg alone, in upper or lower chamber, did no significantly inhibit subset proliferation, in the presence of exogenous ATP we observed a strong inhibition of CD73⁺ Tconv proliferation located in the upper chamber without affecting the proliferation of CD73^{neg} Tconv (FIG. 2).

Collectively these data highlight the capacity of Treg, to mediate ATP dependent CD73⁺ Tconv suppression through autocrine Ado production. Moreover, this cooperation may occur proximally or remotely without the need of Treg/CD73⁺ Tconv tight co-localization. CD73^{neg} Tconv may also be inhibited only if they are in direct contact with CD73⁺ Tconv generating Ado.

Tumor Environment Contains Highly Functional CD39⁺ Treg and CD73⁺ Tconv with Th1/17 Characteristics

To complete our study, we analyzed human tumors. Firstly, we found that Breast tumor (BT) and Ovarian Tumor (OT) patients' blood compared to HD blood, display similar characteristic in terms of CD39/CD73 expression on Treg and on Tconv (data not shown). Percentage of Treg in BT-Blood (Mean±SEM: 6.36±2.47%) and OT Blood (Mean±SEM: 4.09±2.46%) were not different from than those observed in HD-Blood (Mean±SEM: 4.34±1.14%) but, in contrast to CD39⁺ Tconv, the percentage of CD39⁺ cells and the intensity of CD39 these cells among Treg tended to increase in BT-Blood (Mean±SEM: 62.31±14.94%) and OT-Blood (Mean±SEM: 62.79±14.93%) compared to HD-Blood (Mean±SEM: 49.36±23.60%). In contrast, there was no modulation in term of frequency of and function (cytokines production, Rhd123 efflux) of CD73⁺ Tconv (or CD73^{neg} Tconv) in patients' Blood.

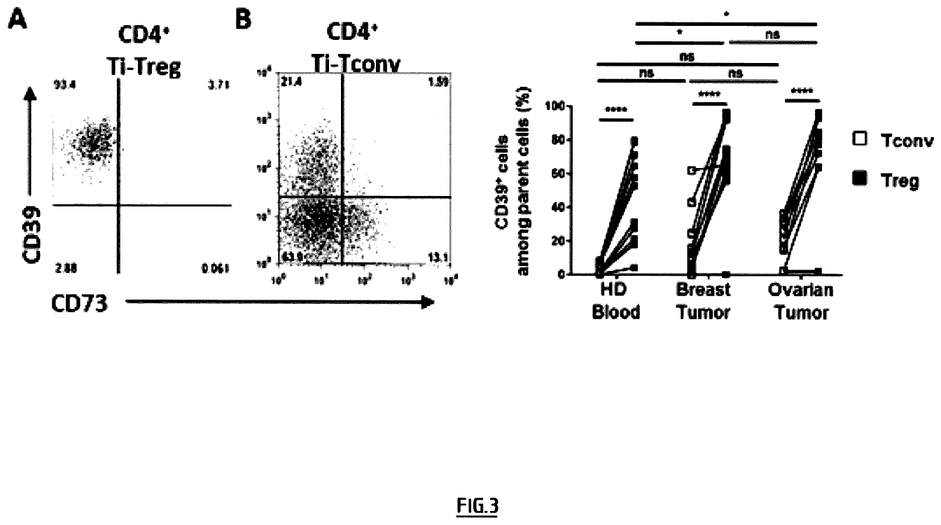

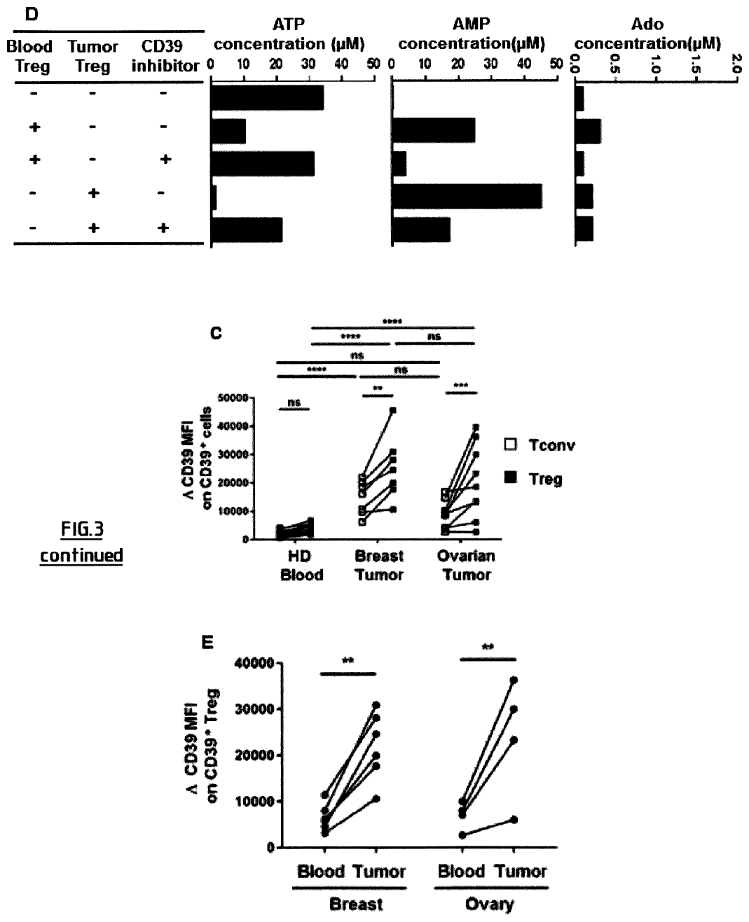

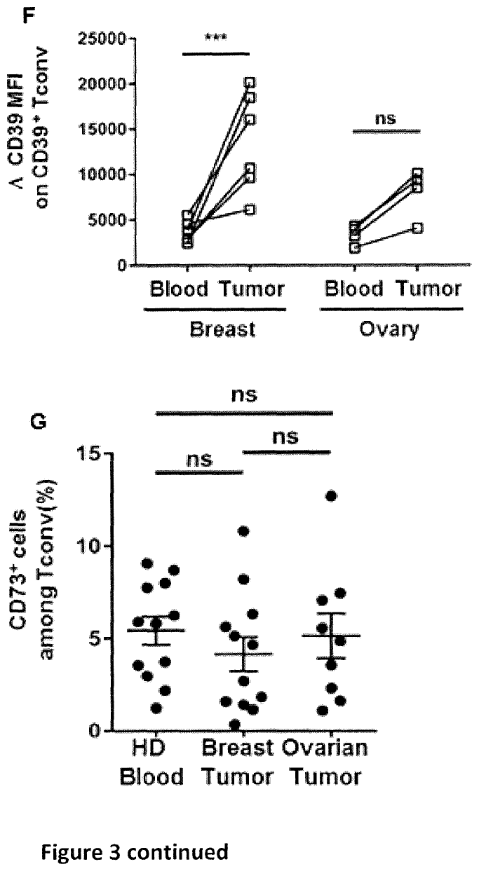

In tumor tissues, we found, as previously reported by our team and others, higher percentage of Treg among memory CD4⁺ T cells in breast (Mean±SEM: 17.95±8.96%) and ovarian (Mean±SEM: 20.92±10.22%) tumor environment compared to blood from either HD % (Mean±SEM: 49.36±23.60%) or patients (BT-Blood Mean±SEM: 6.36±2.47%; OT-Blood: Mean±SEM: 4.09±2.46%). Similar observations were done in colorectal cancer (CRC) tissues (Mean±SEM: 12.29%) compared to healthy colonic tissues (6.42±1.81%). In BT and OT environment, as observed in blood, infiltrating Treg expressed only CD39 (FIG. 3A) and this expression on tumor-infiltrating Treg is still higher than on Tconv and increased compared to HD-blood, both in terms of percentage (Mean±SEM HD: 49.36±23.60%, BT: 67.31±15.93% and OT: 70.77±18.19%) (FIG. 3B), and intensity (FIG. 3C). This upregulated CD39 expression translated into an increased capacity to degrade exogenous ATP into AMP but not Ado and Ino (FIG. 3D). Interestingly, analysis of patient's blood and associated BT or OT revealed a higher CD39 intensity of on Treg (and Tconv) from tumor compared to peripheral blood (FIG. 3E, F). Percentage of CD39⁺ Treg was also significantly increased in CRC tissue (Mean±SEM: 65.35±17.11%) compared to healthy colonic tissue (Mean±SEM: 43.67±19.92%).

In contrast CD73⁺ Tconv were also detected in tumor tissue (FIG. 3A) and their frequency was not significantly modulated in tumor environment (Mean±SEM BT: 4.74±2.64%, OT: 4.73±2.71%) compared to blood (Mean±SEM HD-Blood: 3.62±2.24%) (FIG. 3G).

Within breast and ovarian tumor environment, as observed in blood, higher percentage of IL-2, TNF-α, IFN-γ and IL-17A producing, and IFN-γ/IL-17A co-producing, cells were found among CD73⁺ Tconv compared to CD73^{neg} Tconv. We noticed a significant decrease of TNF-α and IL-2 producing cells among CD73⁺ and CD73^{neg} Tconv in tumor environment compared to blood. Interestingly, IL-17A and IFN-γ/IL-17A coproducing cells in CD73⁺ Tconv, in contrast to CD73^{neg} Tconv were decreased in BT, but not in OT, compared to HD-Blood whereas percentage of IFN-γ producing cells were not affected.

As our data obtained in blood suggested that targeting of CD4⁺ T cells by Ado may occur through cooperation with CD73⁺ Tconv and Treg for ATP degradation, we evaluated expression of A2aR and A2bR and ADA in tumor and blood memory CD4⁺ T cells by Western Blot. In contrast to blood memory CD4⁺ T cells, tumor-infiltrating Tconv expressed A2aR and A2bR but low levels of ADA suggesting that these cells will be more sensitive to Ado in tumor tissue).

CD73⁺ Tconv do not Express Other Inhibitory Immune Checkpoints

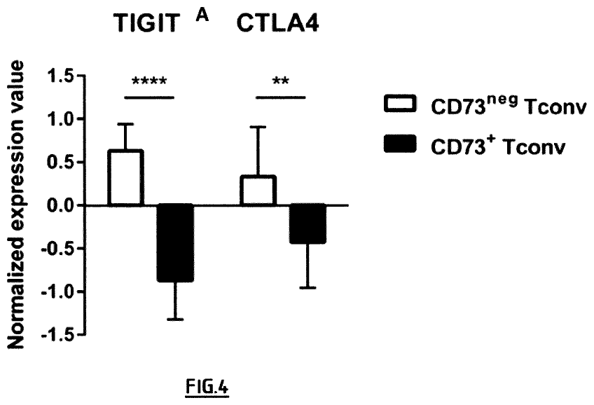

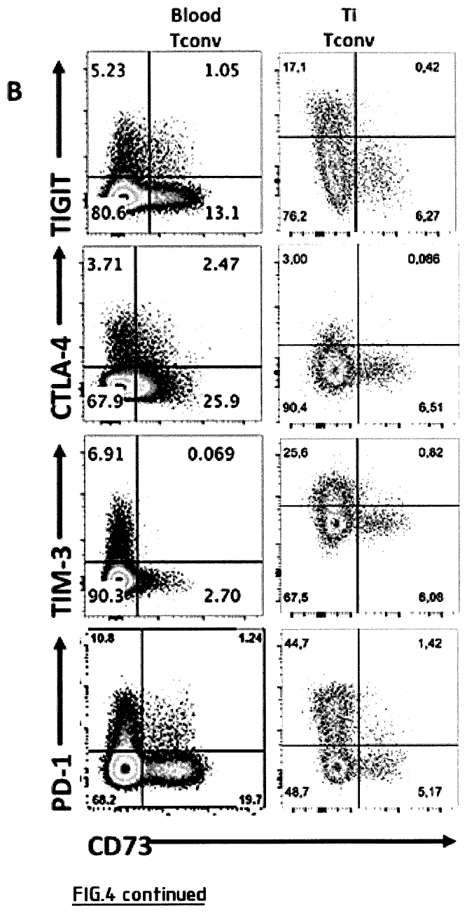

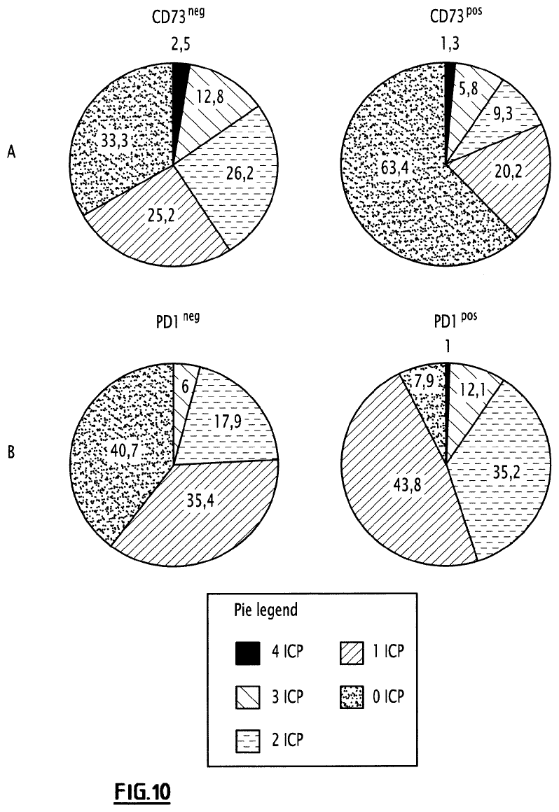

As we demonstrated that CD73 could be considered as a functional regulator of a particular CD4⁺ subset with Th1/17 functional properties, we evaluated the co-expression of other immune checkpoints (ICP). First of all, looking at the transcriptomic analysis led us to highlight that CTLA4 and TIGIT two well-known inhibitory ICP were strongly down-regulated in CD73⁺ subset (TIGIT: fold change (Log 2)=−1.98, p=0.0001; CTLA4: fold change (Log 2)=−1.42, p<10⁻⁴) compared to CD73^{neg} cells (FIG. 4A). We then analyzed by flow cytometry the co-expression of ICP such as TIGIT, CTLA-4, TIM-3 and PD-1 with CD73 on memory CD4⁺ T cells. Interestingly, we observed that CD73⁺ Tconv from either HD-Blood or tumor tissue seems to have a lower expression of ICP than CD73^{neg} Tconv (FIG. 4B). In fact, analysis of inhibitory ICP co-expression on these cells from BT (FIG. 10A) and OT (not shown) showed that CD73⁺ Tconv contained two fold more cells devoid of any inhibitory ICP (63% vs 33.4%), more than two fold less cells co-expressing 2, 3 or 4 ICP (16.4% vs 41.5%) but similar amounts of cells expressing only one ICP compared to CD73^{neg} Tconv (20.2% vs 25.2%). This strongly contrasted with the results obtained with PD-1 as most of the PD-1⁺ Tconv population (92.1%) also co-expressed other inhibitory ICPs (FIG. 10B). Moreover, statistical analysis of individual inhibitory ICP MFI confirmed that CD73⁺ Tconv presented lower staining intensity for TIGIT and PD1, but not for CTLA-4 and TIM-3 than CD73^{neg} Tconv.

Herein the results of this analysis associated with the previous evidence of CD39⁺ Treg selective targeting of CD73⁺ Tconv through Ado generation strongly suggest that human CD73⁺ Tconv associated with Th1/17 characteristics are not regulated through known inhibitory ICP but mainly through the adenosinergic axis.

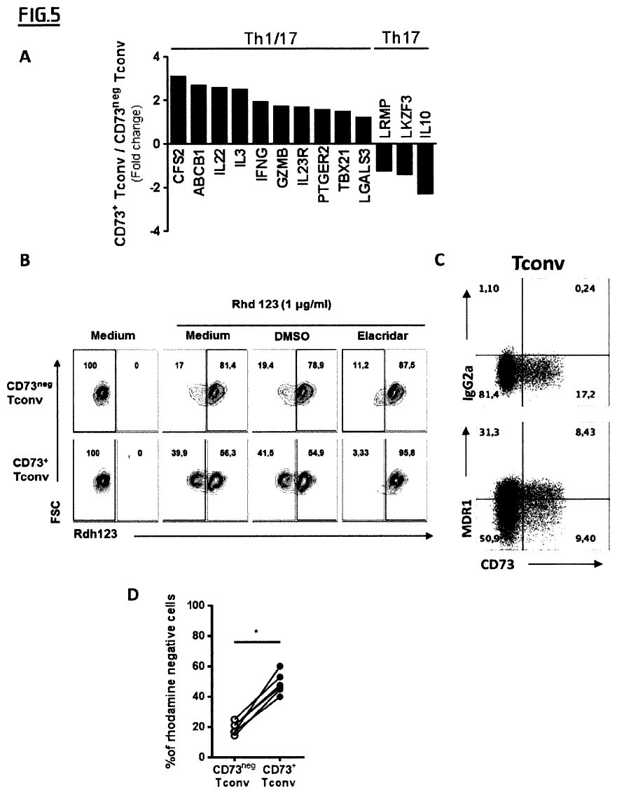

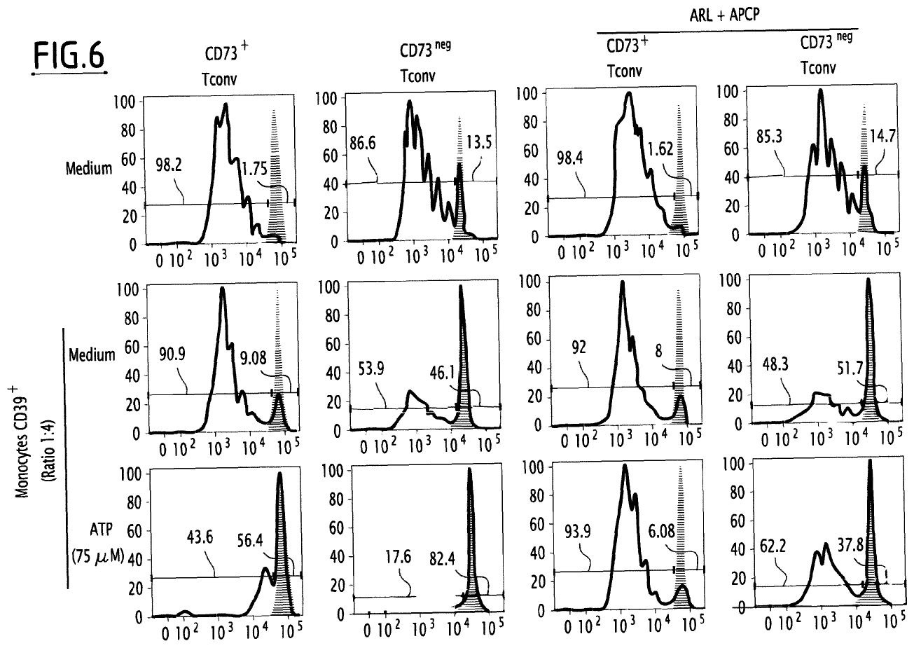

In Presence of Exogenous ATP, CD39+ Monocytes Block Proliferation of Both CD73+ and CD73neg Subsets are but Only CD73+ One Depends on CD39/CD73—See FIG. 6

Coculture with Monocytes blocks CD73neg but not CD73+ proliferation independently from CD73/CD39 axis another pathway involved (PDL-1, IL-10, PGE-2 . . . ).

In presence of exogenous ATP, both subsets are inhibited but only CD73+ inhibition depends on CD73/CD39

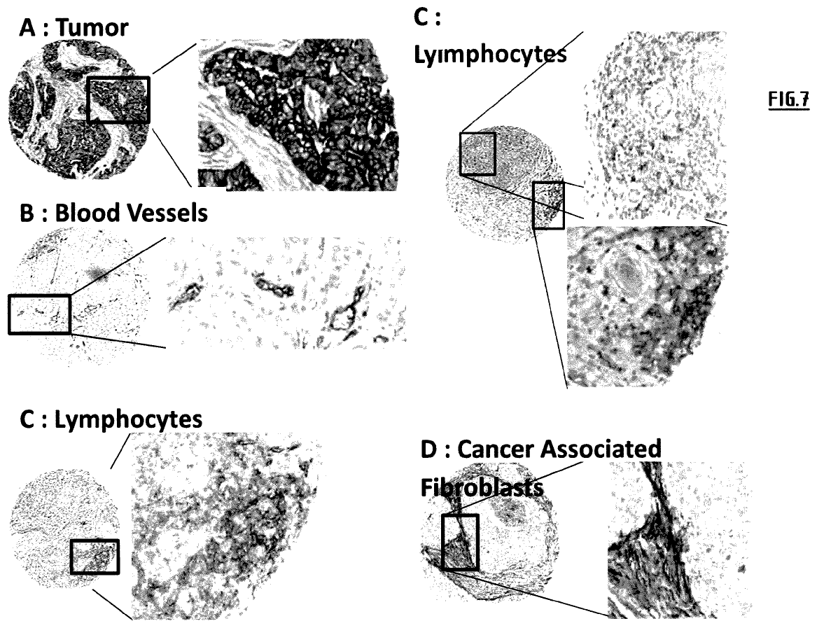

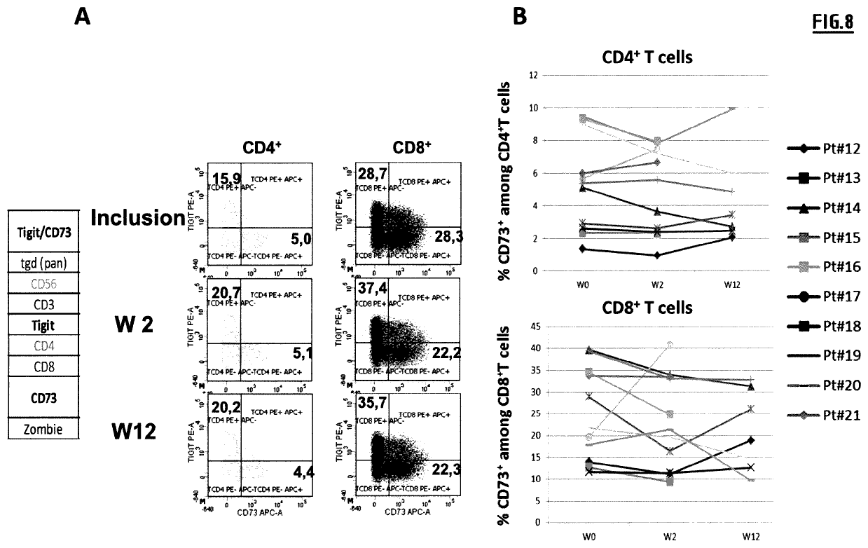

CD73 is not Significantly Modulated on CD4+ T Cells During Anti-PD1 Treatment in Blood of Metastatic Melanoma Patients: See FIG. 8.

1-18. (canceled)

19. A method for selecting a human patient for CD73 antagonist therapy, comprising the step of determining in a patient's biological sample, that lymphocytes express CD73, wherein, upon a determination that lymphocytes express CD73 in patient's biological sample, the patient is declared sensitive to immunotherapy using an antagonist of CD73.

20. The method of claim 1, comprising the determination (i) in a tumor tissue or environment sample whether infiltrating lymphocytes or stromal element contacting lymphocytes express CD73, and/or (ii) determination in blood sample whether lymphocytes express CD73.

21. The method of claim 1, wherein the lymphocytes are CD4+ T cells.

22. The method of claim 1, wherein the immunotherapy comprises administering an anti-CD73 antibody.

23. The method of claim 1, further comprising determining the presence of Treg and/or monocytes expressing CD39.

24. The method of claim 1, further comprising determination of whether said cells expressing CD73 also express MDR1.

25. The method of claim 1, wherein upon said determination of cells expressing CD73 and possibly MDR1, the patient is declared sensitive to combined therapy with an antagonist of CD73 and a chemotherapeutic agent sensitive to MDR1 exclusion activity.

26. The method of claim 1, wherein upon determination that lymphocytes express CD73 in patient's biological sample, and possibly presence of Treg and/or monocytes expressing CD39, conclusion is made that the human patient is at risk of resistance to, or lack of efficacy of, cancer immunotherapy directed against an inhibitory immune checkpoint (ICP).

27. A method of treating a human patient against cancer, wherein the patient comprises CD73-expressing lymphocytes, comprising administering to said patient an effective amount of a CD73 antagonist.

28. The method of claim 27, wherein said human patient has tumor infiltrating lymphocytes or stromal element contacting lymphocytes expressing CD73, and/or blood lymphocytes expressing CD73.

29. The method of claim 27, wherein said patient has Treg and/or monocytes expressing CD39.

30. The method of claim 27, further comprising administering to said patient an effective amount of a chemotherapeutic agent sensitive to MDR1 exclusion activity.

31. A method for assessing in a human patient a risk of resistance to, or lack of efficacy of, cancer immunotherapy directed against an inhibitory immune checkpoint (ICP), comprising the step of determining in a patient's biological sample whether lymphocytes expressing CD73 are present, wherein detecting CD73-expressing lymphocytes is indicative of a risk of resistance to, or lack of efficacy of, cancer immunotherapy directed against said inhibitory ICP.

32. The method of claim 31, wherein immunotherapy is and anti-PD1 or anti-CTLA4 immunotherapy.

33. The method of claim 31, further comprising determining the presence of Treg and/or monocytes expressing CD39.

34. The method of claim 31, wherein upon detecting CD73-expressing lymphocytes, and possibly cells expressing CD39, the patient is declared sensitive to treatment with an antagonist of CD73, in addition to the immunotherapy against said inhibitory ICP.

35. The method of claim 31, further comprising determination of whether said cells expressing CD73 also express MDR1.

36. The method of claim 31, wherein the patient is declared sensitive to treatment with a chemotherapeutic agent sensitive to MDR1 exclusion activity.

37. A method of treating a patient comprising CD73-expressing lymphocytes against cancer, wherein the patient is to be treated or is being treated with a cancer immunotherapy directed against an inhibitory ICP and/or a chemotherapy with a chemotherapeutic agent sensitive to MDR1 exclusion activity, comprising administering to said patient an effective amount of a CD73 antagonist.

38. A kit comprising a CD73 antagonist and a chemotherapeutic agent sensitive to MDR1 exclusion activity, for simultaneous, separate or sequential use to treat cancer in a patient comprising CD73-expressing lymphocytes.
